(19) United States
(12) Patent Application Publication
(10) Pub. No.: US 2023/0210458 A1
(43) Pub. Date: Jul. 6, 2023

(54) Electronics Module

(71) Applicant: Prevayl Innovations Limited, Manchester, Greater Manchester (GB)

(72) Inventor: Michael John Lynch

(21) Appl. No.: 18/000,086

(22) PCT Filed: Jun. 15, 2021
(86) PCT No.: PCT/GB2021/051490

§ 371 Date: Nov. 28, 2022

(30) Foreign Application Priority Data
Jun. 18, 2020 (GB) 2009329.0

(51) Int. Cl. A61B 5/00; G06F 1/16; G06F 3/0346

(52) U.S. Cl. CPC A61B 5/6802; G06F 1/1694; G06F 3/0346; A61B 2562/166

(57) ABSTRACT

The electronics module (100) comprises a housing (101) comprising an opening (17). A processor (109). A flexible electronics structure (500) comprising a flexible substrate on which an electronics component 105 is provided. The electronics component (105) is communicatively connected to the processor 109. The flexible substrate extends through the opening (17) in the housing (101) such that the electronics component (105) is located at least partially outside of the housing (101). The processor is located within the housing.

The present invention is directed towards an electronics module for a wearable article.

### BACKGROUND

Wearable articles can be designed to interface with a wearer of the article, and to determine information such as the wearer’s heart rate, rate of respiration, activity level, and body positioning. Such properties can be measured with a sensor assembly that includes a sensor for signal transduction and/or microprocessors for analysis. The articles include electrically conductive pathways to allow for signal transmission between an electronics module for processing and communication and sensing components of the article. The wearable articles may be garments. Such garments are commonly referred to as ‘smart clothing’ and may also be referred to as ‘biosensing garments’ if they measure biosignals.

It is desirable to overcome at least some of the problems associated with the prior art, whether explicitly discussed herein or otherwise.

### SUMMARY

According to the present disclosure there is provided an electronics module and an apparatus as set forth in the appended claims. Other features of the invention will be apparent from the dependent claims, and the description which follows.

According to a first aspect of the disclosure, there is provided an electronics module for a wearable article. The electronics module comprises a housing comprising an opening; a processor; and a flexible electronics structure comprising a flexible substrate on which an electronics component is provided, wherein the electronics component is communicatively connected to the processor. The flexible substrate extends through the opening in the housing such that the electronics component is located at least partially outside of the housing, and the processor is located within the housing.

The electronics component is electrically coupled to the processor located inside the housing but is located at least partially outside of the housing. This arrangement helps space the sensor away from the processor and into an optimum position. The sensor may be located closer to a skin surface when the electronics module is worn and is spaced from the processor so that it is less affected by heat generated by the processor and/or other elements located within the electronics module.

The electronics module may further comprise a contact pad. The electronics component may be sandwiched between the contact pad and the housing. Sandwiching the electronics component between the contact pad the housing helps protect the sensor component against damage and protects against water ingress into the housing via the opening.

The contact pad may be shaped to accommodate at least part of the electronics component. The contact pad may comprise a recess sized to receive at least part of the electronics component. The contact pad may be in thermal contact with the electronics component. The contact pad may be electrically connected to the processor.

The housing may comprise an opening for receiving at least part of the contact pad. The housing may comprise a first enclosure and a second enclosure which are connected to one another. The first enclosure and the second enclosure may be connected to one another using a snap-fit mechanism.

The electronics component may be attached to an external surface of the housing. The electronics component may be located in a recess provided in an external surface of the housing.

The electronics component may be or comprise a sensor.

According to a second aspect of the disclosure, there is provided a method of assembling an electronics module for a wearable article. The method comprises: providing a housing comprising an opening; providing an assembly comprising a processor and a flexible electronics structure comprising a flexible substrate on which an electronics component is provided, wherein the electronics component is communicatively connected to the processor; and positioning the assembly in the housing such that the flexible substrate extends through the opening in the housing, the electronics component is located at least partially outside of the housing, and the processor is located within the housing.

The flexible electronics structure may comprise a connector interface region which is communicatively connected to the processor. The flexible electronics structure may comprise an end region on which the electronics component is provided. The end region may be able to hang downwards due to gravity.

Positioning the assembly in the housing may comprise lowering the assembly into the housing such that the end region of the flexible electronics structure passes through the opening in the housing.

Providing the housing may comprise providing a first enclosure comprising the opening. The method may further comprise attaching a second enclosure to the first enclosure to form an enclosed space in which the processor is located.

The method may further comprise providing a power source. The method may further comprise attaching the power source to the processor. The attaching may comprise electrically and mechanically attaching the power source to the processor. The power source may be attached to the processor prior to positioning the assembly in the housing.

The method may further comprise attaching a contact pad to an external surface of the housing such that the electronics component is sandwiched between the contact pad and the housing. The method may further comprise locating the electronics component in a recess of the contact pad sized to receive at least part of the electronics component.

The method may further comprise attaching the electronics component to an external surface of the housing. The electronics component may be located in a recess provided in an external surface of the housing.

According to a third aspect of the disclosure, there is provided a contact pad assembly for a wearable article, the contact pad assembly comprising a contact pad and an electronics component located with the contact pad.

The contact pad may comprise a conductive material. The contact pad may comprise an elastomeric material. The contact pad may comprise a conductive elastomeric material.

The electronics component may be attached to the contact pad. The electronics component may be overmoulded with the contact pad.

The contact pad may be shaped to accommodate the electronics component. The contact pad may comprise a recess in which the electronics component is at least partially located.

The contact pad assembly may further comprise a connector arranged to connect the electronics component with a further electronics component of the wearable article. The connector and the electronics component may be provided on the same flexible substrate.

The flexible substrate may comprise a stiffener material. The stiffener material may be provided in the vicinity of one or both of the electronics component and the connector.

The contact pad may be arranged to interface with a connector of the wearable article so as to bring a further electronics component of the wearable article into communication with the contact pad. The contact pad may be shaped to accommodate the connector. The contact pad may comprise a projection extending from a surface of the contact pad to interface with the connector.

The contact pad may comprise a surface arranged to interface with an external component so as to couple signals between the external component and a processor of the wearable article. The surface may have a three-dimensional texture.

The electronics component may comprise a sensor. The sensor may comprise a temperature sensor.

The electronics component may be in physical contact with the contact pad.

According to a fourth aspect of the disclosure, there is provided an electronics module for a wearable article. The electronics module comprises a processor and a contact pad assembly of the third aspect of the disclosure.

The contact pad assembly may be spaced apart from the processor.

The electronics module may comprise a housing. The processor may be disposed within the housing and the contact pad assembly may be located at least partially outside of the housing. The electronics component may be sandwiched between the contact pad and the housing.

The contact pad may be communicatively coupled to the processor.

According to a fifth aspect of the disclosure, there is provided a contact pad for a wearable article. The contact pad is shaped to accommodate an electronics component.

According to a sixth aspect of the disclosure, there is provided an electronics module for a wearable article, the electronics module comprising a processor and a contact pad of the fifth aspect of the disclosure.

According to a seventh aspect of the disclosure, there is provided a flexible electronics structure for a wearable article comprising: a flexible substrate material comprises a first arm and a second arm, wherein the first arm and the second arm are moveable relative to one another; a first electronics component provided on the first arm; and a second electronics component provided on the second arm.

The first electronics component may comprise a sensor. The sensor may comprise a temperature sensor.

The second electronics component may comprise a radio-frequency antenna. The radio-frequency antenna may be formed from conductive traces provided around an aperture formed in the second arm.

The flexible electronics structure may further comprise a shared interface region, wherein the first electronics component and the second electronics component are electrically connected to the shared interface region.

The flexible electronics structure may further comprise a stiffener material. The stiffener material may be applied to the first arm.

The first electronics component may be provided in an end region of the first arm. The end region of the first arm has a round or pointed shape.

According to an eighth aspect of the present disclosure, there is provided an electronics module for a wearable article, the electronics module comprising the flexible electronics structure of the seventh aspect of the disclosure and a processor.

The flexible electronics structure may comprise a shared interface region, wherein the first electronics component and the second electronics component are electrically connected to the shared interface region, and wherein the processor is electrically connected to the flexible electronics structure via the shared interface region.

The electronics module may comprise a printed circuit board on which the processor is provided, and wherein the printed circuit board comprises a connector interface for connecting with the shared interface region of the flexible electronics structure.

The printed circuit board may comprise a cut-out region in the vicinity of the connector interface sized to accommodate a bend in the flexible electronics structure.

The electronics module may further comprise a housing, wherein the processor is disposed in the housing, and the flexible electronics structure is disposed partially outside of the housing.

The second electronics component may be disposed within the housing and the first electronics component may be disposed at least partially outside of the housing.

According to a ninth aspect of the disclosure, there is provided a flexible electronics structure for a wearable article, comprising: a flexible substrate material on which a first electronics component and a second electronics component are provided, and a shared interface region, wherein the first electronics component and the second electronics component are electrically connected to the shared interface region.

According to a tenth aspect of the disclosure, there is provided a printed circuit board assembly for a wearable article, the printed circuit board assembly comprising a printed circuit board arranged to be electrically connected with a flexible electronics structure comprising a flexible substrate, the printed circuit board further comprising a cut-out region, wherein the cut-out region is shaped to accommodate a bend in the flexible substrate.

The cut-out region may be provided in the vicinity of the electrical connection with the flexible electronics structure.

The printed circuit board assembly may comprise a connector interface for electrically connected with the flexible electronics structure. The cut-out-region may be provided in the vicinity of the connector interface.

The connector interface may enable a removable mechanical and electrical connection between the printed circuit board and the flexible electronics structure. This is not required in all aspects of the disclosure. The printed circuit board and the flexible electronics structure may be permanently mechanically and electrically connected together. The printed circuit board and the flexible electronics structure may be connected together by hot bar solder. The printed circuit board and the flexible electronics structure may form an integral structure such as a rigid-flex printed circuit board. The printed circuit board may be a rigid component of the rigid-flex printed circuit board. One or more conductive traces extend from the printed circuit board the flexible electronics structure to form the electrical connection between the printed circuit board and the flexible electronics structure.

The printed circuit board assembly may further comprise an interface element arranged to interface with a contact pad. The interface element may comprise a force-biased conductor. The interface element may be arranged to receive signals from a further component via the contact pad.

The printed circuit board assembly may further comprise a processor arranged to process signals received via the interface element. The processor may be provided on the printed circuit board. The printed circuit board assembly may further comprise a communicator. The light source may be provided on the printed circuit board. The printed circuit board assembly may further comprise a light source. The light source may be provided on the printed circuit board.

The printed circuit board assembly may further comprise the flexible electronics structure comprising the flexible structure, wherein the flexible electronics structure is electrically connected to the printed circuit board. The flexible electronics structure may be the flexible electronics structure of the eighth or ninth aspect of the disclosure.

According to an eleventh aspect of the present disclosure, there is provided an electronics module for a wearable article. The electronics module comprises a housing, an antenna comprising an aperture and a light source positioned below the antenna and able to emit light through the aperture, wherein the housing comprises a region of localised thinning aligned with the aperture such that light emitted by the light source is able to pass through the region of localised thinning.

The region of localised thinning may be surrounded by a region of increased wall thickness. The region of localised thinning and the region of increased wall thickness may be able to be received within the aperture.

The region of localised thinning may be the same size as or smaller than the aperture.

The region of localised thinning may be integrally formed with the remainder of the housing.

The region of localised thinning and the remainder of the housing may be formed from the same material. The region of localised thinning and the remainder of the housing may be formed together using an injection moulding process. The region of localised thinning may have a thickness of between a half and a quarter of the wall thickness of the remainder of the housing. The region of localised thinning may have a thickness of a third of the wall thickness of the remainder of the housing.

The electronics module may further comprise a printed circuit board on which the light source is provided.

The electronics module may further comprise an interface arranged to communicatively couple the electronics module with a further electronics component. The further electronics component may be a sensing component of the wearable article.

The wearable article may comprise one or more sensing components. The sensing components may be biosensing components. The sensing components may comprise one or more components of a temperature sensor, a humidity sensor, a motion sensor, an electropotential sensor, an electroimpedance sensor, an optical sensor, an acoustic sensor. Here, “component” means that not all of the components of the sensor may be provided in the wearable article. The processing logic, power and other functionality may be provided in the electronics module. The wearable article may only comprise the minimal functionality to perform the sensing such as by only including sensing electrodes. The temperature sensor may be arranged to measure an ambient temperature, a skin temperature of a human or animal body, or a core temperature of a human or animal body. The humidity sensor may be arranged to measure humidity or skin-surface moisture levels for a human or animal body. The motion sensor may comprise one or more of an accelerometer, a gyroscope, and a magnetometer sensor. The motion sensor may comprise an inertial measurement unit. The electropotential sensor may be arranged to perform one or more bioelectrical measurements. The electropotential sensor may comprise one or more of electrocardiography (ECG) sensor modules, electrogastrography (EGG) sensor modules, electroencephalography (EEG) sensor modules, and electromyography (EMG) sensor modules. The electroimpedance sensor may be arranged to perform one or more bioimpedance measurements. Bioimpedance sensors can include one or more of plethysmography sensor modules (e.g., for respiration), body composition sensor modules (e.g., hydration, fat, etc.), and electroimpedance tomography (EIT) sensors. An optical sensor may comprise a photoplethysmography (PPG) sensor module or an orthopantomogram (OPG) sensor module.

The present disclosure is not limited to wearable articles. The electronics arrangement disclosed herein may be incorporated into other forms of devices such as user electronic devices (e.g. mobile phones). In additions, they may be incorporated into any form of textile article. Textile articles may include upholstery, such as upholstery that may be positioned on pieces of furniture, vehicle seating, as wall or ceiling decor, among other examples.

### BRIEF DESCRIPTION OF THE DRAWINGS

Examples of the present disclosure will now be described with reference to the accompanying drawings, in which:

FIG. 1 shows a schematic diagram for an example system according to aspects of the present disclosure;

FIG. 2 shows a sectional view of an example apparatus comprising an electronics module and a wearable article according to aspects of the present disclosure;

FIG. 3 shows a schematic diagram for an example electronics module according to aspects of the present disclosure;

FIGS. 4 and 5 show perspective views of an example assembled electronics module according to aspects of the present disclosure;

FIG. 6 shows a perspective view of the electronics module of FIGS. 4 and 5 with the housing and contact pads removed;

FIG. 7 shows a perspective view of the electronics module of FIGS. 4 and 5 with the housing removed;

FIG. 8 shows a view from the side of the electronics module of FIGS. 4 and 5 with the housing removed;

FIG. 9 shows a view from below of the electronics module of FIGS. 4 and 5 with the housing removed. One of the contact pads is rendered transparent so that the components covered by the contact pad are visible;

FIG. 10 shows a perspective view the electronics module of FIGS. 4 and 5 with the housing and power source removed;

FIG. 11 shows a perspective view of a printed circuit board assembly of the electronics module of FIGS. 4 and 5;

FIG. 12 shows a plan view of a flexible electronics structure of the electronics module of FIGS. 4 and 5;

FIG. 13 shows a perspective view of an assembly comprising the flexible electronics structure of FIG. 12 coupled to the printed circuit board assembly of FIG. 11 during a stage in the process of assembling the electronics module;

FIG. 14 shows a perspective view of an assembly comprising a power source attached to the assembly of FIG. 13 during a stage in the process of assembling the electronics module;

FIG. 15 shows a perspective view of the assembly of FIG. 14 in the process of being lowered into the bottom enclosure of the housing. The bottom enclosure is rendered transparent so that the components within the bottom enclosure are visible;

FIGS. 16 and 17 show the underside surface of the bottom enclosure after the assembly of FIG. 14 has been lowered into the bottom enclosure;

FIG. 18 shows a perspective view of a contact pad of the electronics module of FIGS. 4 and 5 in isolation;

FIG. 19 shows the contact pad of FIG. 18 from above;

FIG. 20 shows the contact pad of FIG. 18 from below;

FIG. 21 shows a perspective view of a contact pad assembly of the electronics module of FIGS. 4 and 5 in isolation;

FIG. 22 shows a close-up view of part of the inner surface of the bottom enclosure of the electronics module of FIGS. 4 and 5. A part of the contact pad that has extended into the bottom enclosure is visible;

FIG. 23 shows a close-up view of the inner surface of the top enclosure of the electronics module of FIGS. 4 and 5;

FIG. 24 shows a sectional view through the top enclosure of FIG. 23;

FIG. 25 shows an exploded view of the top enclosure of FIG. 23 and an antenna disposed within the electronics module;

FIG. 26 shows another example flexible electronics structure according to aspects of the present disclosure;

FIG. 27 shows a partially assembled view of another example electronics module according to aspects of the present disclosure; and

FIG. 28 shows a flow diagram for an example method of assembling an electronics module according to aspects of the present disclosure.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not forthe purpose of limiting the disclosure as defined by the appended claims and their equivalents.

It is to be understood that the singular forms “a,” “an,” and “the” include plural referents unless the context clearly dictates otherwise.

“Wearable article” as referred to throughout the present disclosure may refer to any form of electronic device which may be worn by a user such as a smart watch, necklace, bracelet, headphones, in-ear headphones, or glasses. The wearable article may be a textile article. The wearable article may be a garment. The garment may refer to an item of clothing or apparel. The garment may be a top. The top may be a shirt, t-shirt, blouse, sweater, jacket/coat, or vest. The garment may be a dress, brassiere, shorts, pants, arm or leg sleeve, vest, jacket/coat, glove, armband, underwear, headband, hat/cap, collar, wristband, stocking, sock, or shoe, athletic clothing, swimwear, personal protection equipment, wetsuit or drysuit.

The wearable article/garment may be constructed from a woven or a non-woven material. The wearable article/garment may be constructed from natural fibres, synthetic fibres, or a natural fibre blended with one or more other materials which can be natural or synthetic. The yarn may be cotton. The cotton may be blended with polyester and/or viscose and/or polyamide according to the particular application. Silk may also be used as the natural fibre. Cellulose, wool, hemp and jute are also natural fibres that may be used in the wearable article/garment. Polyester, polycotton, nylon and viscose are synthetic fibres that may be used in the wearable article/garment.

The garment may be a tight-fitting garment. Beneficially, a tight-fitting garment helps ensure that the sensor devices of the garment are held in contact with or in the proximity of a skin surface of the wearer. The garment may be a compression garment. The garment may be an athletic garment such as an elastomeric athletic garment. The present disclosure is not limited to wearable articles for humans and includes wearable articles for animals such as animal collars, jackets and sleeves.

The following description refers to particular examples of the present disclosure where the wearable article is a garment. It will be appreciated that the present disclosure is not limited to garments and other forms of wearable article are within the scope of the present disclosure as outlined above.

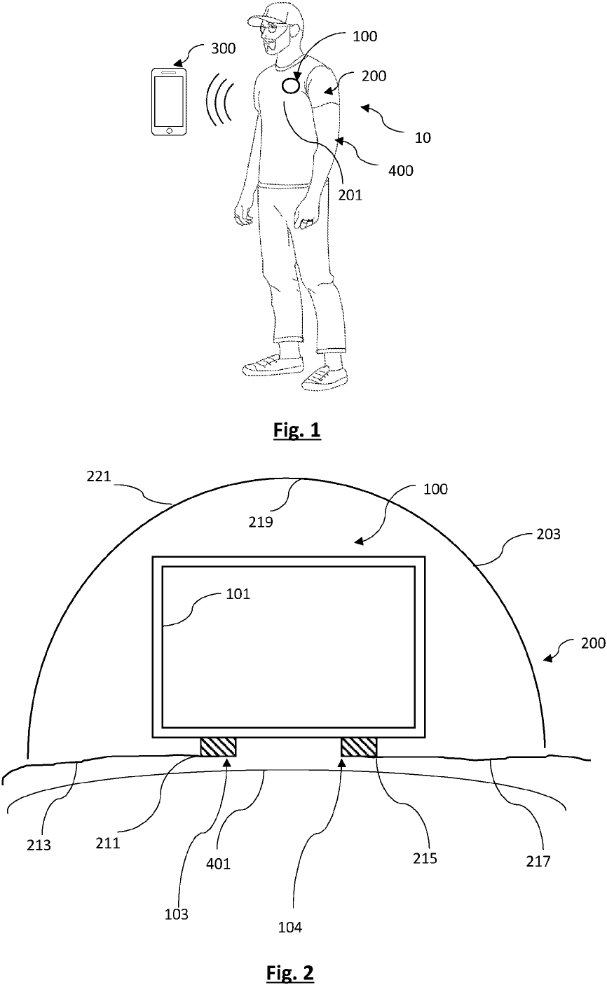

Referring to FIG. 1, there is shown an example system 10 according to aspects of the present disclosure. The system 10 comprises an electronics module 100, a garment 200, and a mobile device 300. The garment 200 is worn by a user 400. The electronics module 100 is attached to the garment 200. The electronics module 100 is shown on the outside surface 201 of the garment 200 in FIG. 1 but may also be within the garment 200 or hidden within a pocket or similar mounting arrangement of the garment 200.

The electronics module 100 is arranged to integrate with sensing components incorporated into the garment 200 so as to obtain signals from the sensing components. The sensing components may comprise electrodes. The electronics module 100 is further arranged to wirelessly communicate data to the mobile device 300. Various protocols enable wireless communication between the electronics module 100 and the mobile device 300. Example communication protocols include Bluetooth ®, Bluetooth ® Low Energy, and near-field communication (NFC). In some examples, the electronics module 100 may communicate over a long-range wireless communication protocol.

The electronics module 100 may be removable from the garment 200. The mechanical coupling of the electronic module 100 to the garment 200 may be provided by a mechanical interface such as a clip, a plug and socket arrangement, etc. The mechanical coupling or mechanical interface may be configured to maintain the electronic module 100 in a particular orientation with respect to the garment 200 when the electronic module 100 is coupled to the garment 200. This may be beneficial in ensuring that the electronic module 100 is securely held in place with respect to the garment 200 and/or that any electronic coupling of the electronic module 100 and the garment 200 (or a component of the garment 200) can be optimized. The mechanical coupling may be maintained using friction or using a positively engaging mechanism, for example.

Beneficially, the removable electronic module 100 may contain all of the components required for data transmission and processing such that the garment 200 only comprises the sensor components and communication pathways. In this way, manufacture of the garment 200 may be simplified. In addition, it may be easier to clean a garment 200 which has fewer electronic components attached thereto or incorporated therein. Furthermore, the removable electronic module 100 may be easier to maintain and/or troubleshoot than embedded electronics. The electronic module 100 may comprise flexible electronics such as a flexible printed circuit (FPC). The electronic module 100 may be configured to be electrically coupled to the garment 200.

It may be desirable to avoid direct contact of the electronic module 100 with the wearer’s skin while the garment 200 is being worn. It may be desirable to avoid the electronic module 100 coming into contact with sweat or moisture on the wearer’s skin or other sources of moisture such as from rain or a shower. It may further be desirable to provide an electronics module holder such as a pocket in the garment to contain the electronic module 100 in order to prevent chafing or rubbing and thereby improve comfort for the wearer. The pocket may be provided with a waterproof lining in order to prevent the electronic module 100 from coming into contact with moisture.

Referring to FIG. 2, there is shown a sectional view of an apparatus comprising a garment 200 and an electronics module 100 disposed within an electronics module holder 203 of the garment 200. The garment 200 is being worn by a user and is proximate to the skin surface 401 of the user.

The electronics module holder 203 in this example is an elasticated pocket 203 positioned on the outside surface of the garment 200. In other examples, the electronics module holder 203 may be provided within the garment 200 such as in the form of an inner pocket.

The pocket 203 allows the user to position the electronics module 100 in the pocket 203 and remove it therefrom. The pocket 203 applies a compressive force to help hold the electronics module 100 in a generally fixed position within the pocket 203. This is not required in all examples as gripping surfaces of the electronics module 100 and/or the garment 200/pocket 203 may be sufficient for limiting relative movement between the electronics module 100 and the garment 200. Additionally or separately, the electronics module 100 and the garment 200 may comprise magnetic elements to help hold the electronics module 100 in a fixed position relative to the garment 200. The housing of the electronics module 100 may be constructed to enable a magnet to be retained therein. In particular, a recess may be provided in an inner surface of a bottom enclosure of an electronics module 100 sized to retain a magnet.

The pocket 203 comprises a layer of material 203 which is bonded, stitched, otherwise attached to or integrally formed with the garment 200. The pocket 203 has an inner surface 219 facing the electronics module 100. The pocket 203 has an outer surface 221 which can be considered as part of the outer surface 201, 221 of the garment 200.

The electronics module 100 comprises a housing 101 formed of a rigid material in this example. One or more electrical components are provided within the rigid housing 101. The housing 101 may comprise a (rigid) polymeric material. The polymeric material may be a rigid plastic material. The rigid plastic material may be ABS or polycarbonate plastic, but is not limited to these examples. The rigid plastic material may be glass reinforced. The rigid housing 101 may be injection moulded. The rigid housing 101 may be constructed using a twin-shot injection moulding approach.

A plurality (two in this example) of contact pads 103, 104 are provided on the outer surface of the housing 101. The contact pads 103, 104 are formed from a flexible material, but this is not required in all examples. The contact pads 103, 104 are spaced apart from one another on the bottom surface of the housing 101. “Rigid” will be understood as referring to a material which is stiffer and less able to bend than the contact pads 103, 104 formed of flexible material. The rigid housing 101 may still have some degree of flexibility but is less flexible than the flexible material of the contact pads 103, 104.

The contact pads 103, 104 comprise conductive material, and thus acts as conductive contact pads 103, 104 forthe electronics module 100. The flexible conductors 103, 103 therefore provide the interface by which the electronics module 100 is able to receive signals from an external component such as the garment 200.

The first electrical contact 103 conductively connects with a first terminal region 211 of the garment 200. The first terminal region 211 enables the electronics module 100 to conductively connect to sensing components of the garment 200 via first electrically conductive pathway 213 of the garment 200. The second electrical contact 104 conductively connects with a second terminal region 215 of the garment 200. The second terminal region 215 enables the electronics module 100 to conductively connect to sensing components of the garment 200 via second electrically conductive pathway 217 of the garment 200. The sensing components may be one or more electrodes.

The electrically conductive pathways 213, 217 and terminal regions 211, 215 may be formed from any form of conductive material such as conductive thread or wire. The conductive thread or wire may be woven or otherwise incorporated into a tape or fabric panel. The electrically conductive pathways 213, 217 and terminal regions 211, 215 may be electrically conductive tracks or films. The electrically conductive pathways 213, 217 and terminal regions 211, 215 may be conductive transfers. The conductive material may be formed from a fibre or yarn of the textile. This may mean that an electrically conductive materials are incorporated into the fibre/yarn. The conductive material may be a conductive rubber.

The use of flexible conductors 103, 104 is generally preferred as compared to rigid, metallic, conductors 103, 104 as this means that hard pieces of conductive metallic material such as poppers or studs are not required to electrically connect the electronics module 100 to the garment 200. This not only improves the look and feel of the garment 200 but also reduces manufacturing costs as it means that hardware features such as additional eyelets and studs do not need to be incorporated into the garment 200 to provide the required connectivity. An additional problem with rigid metallic conductors is that their hard, abrasive, surfaces may rub against conductive elements such as conductive thread of the garment and cause the conductive thread to fray.

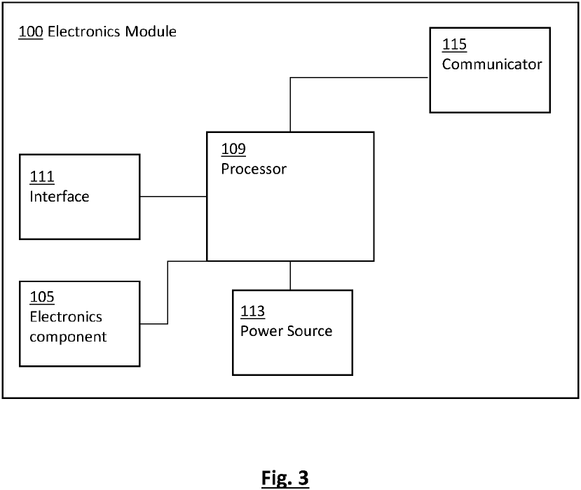

Referring to FIG. 3, there is shown a schematic diagram for an example electronics module 100 according to aspects of the present disclosure.

The electronics module 100 comprises a processor 109 configured to process signals sensed by a sensing component of the electronics module 100 and/or the garment 200. The signals relate to the activity of a user wearing the garment 200.

The electronics module 100 comprises an electronics component 105. The electronics component 105 may comprise an output unit such as a light source or haptic feedback unit. The light source may be arranged to emit light to indicate a status of the electronics module 100 or a property of a user wearing the wearable article, for example. The electronics component 105 may comprise a sensor. The sensor may be arranged to monitor a property of the user. The sensor may be, for example, a temperature sensor arranged to monitor a core body temperature or skin-surface temperature of the user. The sensor may be, for example, a humidity sensor arranged to monitor a hydration or sweat level of the user. The sensor may be a temperature sensor arranged to measure the skin temperature of the user wearing the garment. The temperature sensor may be a contact temperature sensor or a non-contact temperature sensor such as an infrared thermometer. Example contact temperature sensors include thermocouples and thermistors. The sensor may comprise an altitude sensor, presence sensor, or air quality sensor. The presence sensor may for detecting a touch input from a user. The presence sensor may comprise one or more of a capacitive sensor, inductive sensor, and ultrasonic sensor. Other examples of sensor are provided throughout this specification.

The electronics module 100 comprises a power source 113. The power source 113 is coupled to the processor 109 and is arranged to supply power to the processor 109. The power source 113 may comprise a plurality of power sources. The power source 113 may be a battery. The battery may be a rechargeable battery. The battery may be a rechargeable battery adapted to be charged wirelessly such as by inductive charging. The power source 113 may comprise an energy harvesting device. The energy harvesting device may be configured to generate electric power signals in response to kinetic events such as kinetic events performed by a wearer of the garment. The kinetic event could include walking, running, exercising or respiration of the wearer. The energy harvesting material may comprise a piezoelectric material which generates electricity in response to mechanical deformation of the converter. The energy harvesting device may harvest energy from body heat of a wearer of the garment. The energy harvesting device may be a thermoelectric energy harvesting device. The power source may be a super capacitor, or an energy cell.

The power source 113 in this example is a lithium polymer battery 113. The battery 113 is rechargeable and charged via a USB C input of the electronics module 100. Of course, the present disclosure is not limited to recharging via USB and instead other forms of charging such as inductive of far field wireless charging are within the scope of the present disclosure. Additional battery management functionality is provided in terms of a charge controller, battery monitor and regulator. These components may be provided through use of a dedicated power management integrated circuit (PMIC). The processor 109 is communicatively connected to the battery monitor such that the processor 109 may obtain information about the state of charge of the battery 113.

The communicator 115 may be a mobile/cellular communicator operable to communicate the data wirelessly via one or more base stations. The communicator 115 may provide wireless communication capabilities for the garment 200 and enables the garment 200 to communicate via one or more wireless communication protocols such as used for communication over: a wireless wide area network (WWAN), a wireless metroarea network (WMAN), a wireless local area network (WLAN), a wireless personal area network (WPAN), Bluetooth ® Low Energy, Bluetooth ® Mesh, Bluetooth ® 5, Thread, Zigbee, IEEE 802.15.4, Ant, a near field communication (NFC), a Global Navigation Satellite System (GNSS), a cellular communication network, or any other electromagnetic RF communication protocol. The cellular communication network may be a fourth generation (4G) LTE, LTE Advanced (LTE-A), LTE Cat-M1, LTE Cat-M2, NB-IoT, fifth generation (5G), sixth generation (6G), and/or any other present or future developed cellular wireless network. A plurality of communicators may be provided for communicating over a combination of different communication protocols.

The electronics module 100 may comprise a Universal Integrated Circuit Card (UICC) that enables the electronics module 100 to access services provided by a mobile network operator (MNO) or virtual mobile network operator (VMNO). The UICC may include at least a read-only memory (ROM) configured to store an MNO/VMNO profile that the wearable article can utilize to register and interact with an MNO/VMNO. The UICC may be in the form of a Subscriber Identity Module (SIM) card. The electronics module 100 may have a receiving section arranged to receive the SIM card. In other examples, the UICC is embedded directly into a controller of the electronics module 100. That is, the UICC may be an electronic/embedded UICC (eUICC). A eUICC is beneficial as it removes the need to store a number of MNO profiles, i.e. electronic Subscriber Identity Modules (eSIMs). Moreover, eSIMs can be remotely provisioned to electronics modules 100. The electronics modules 100 may comprise a secure element that represents an embedded Universal Integrated Circuit Card (eUICC).

The interface 111 is arranged to communicatively couple with a sensing component of the garment 200 (FIG. 1) so as to receive a signal from the sensing component or may directly interface with a skin surface of the wearer to receive signals therefrom. The processor 109 is communicatively coupled to the interface 111 and is arranged to receive the signals from the interface 111.The interface 111 may form a conductive coupling or a wireless (e.g. inductive) communication coupling with the electronics components of the garment 200. The interface 111 may comprise the contact pads 103, 104 of FIG. 2, for example.

The electronics module 100 is mounted on a garment 200 (FIG. 1) and conductively connected to sensing components such as electrodes of the garment via electrically conductive pathways of the garment 200. In a particular example, the sensing components are electrodes used to measure electro potential signals such as electrocardiogram (ECG) signals.

The processor 109 may be a component of a controller such as a microcontroller. The controller may have an integral communicator such as a Bluetooth ® antenna. The controller may have an internal memory and may also be communicatively connected to an external memory of the electronics module such as a NAND Flash memory. The memory is used to for the storage of data when no wireless connection is available between the electronics module 100 a mobile device 300 (FIG. 1). The processor 109 is connected to the interface 111, 103, 104 via an analog-to-digital converter (ADC) front-end and an electrostatic discharge (ESD) protection circuit. The ADC front-end end converts the raw analog signal received from sensing components of the garment 200 into a digital signal. The ADC front-end may also perform filtering operations on the received signals.

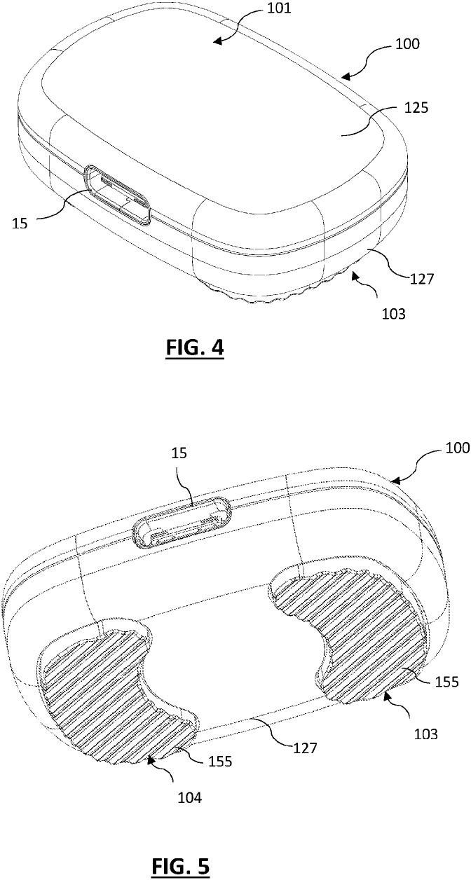

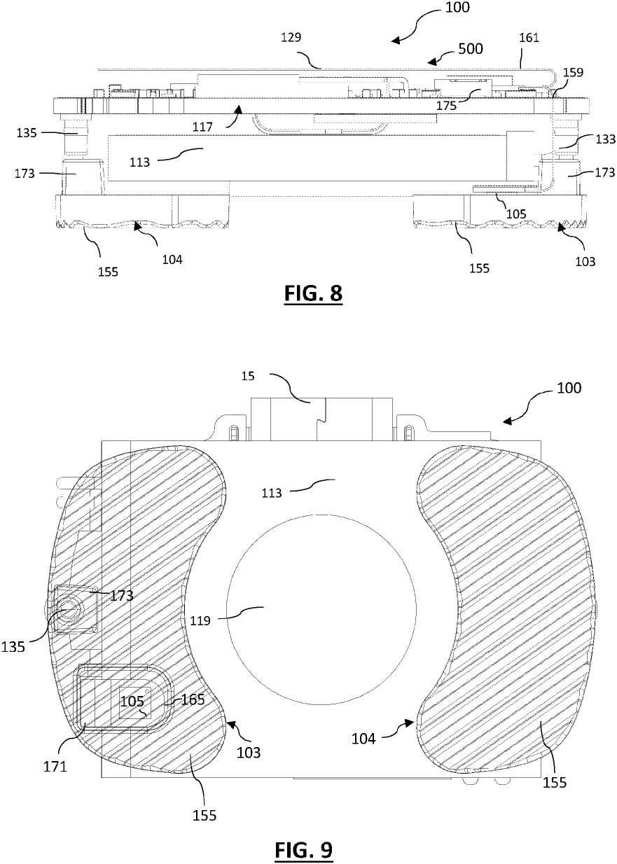

FIGS. 4 to 27 show an example electronics module 100 according to aspects of the present disclosure.

FIGS. 4 and 5 show the electronics module 100 in an assembled state. The electronics module 100 comprises a rigid housing 101 and a plurality (two in this example) of contact pads 103, 104 that are attached to an external surface of the rigid housing 101 and spaced apart from one another. The contact pads 103, 104 in this example are constructed from a flexible material, and in particular a flexible conductive material. The contact pads 103, 104 therefore form an outer layer of flexible material that covers a part of the rigid housing 101. Rigid contact pads 103 such as those made from a rigid metallic material are also within the scope of the present disclosure.

The rigid housing 101 comprises a top enclosure 125 and a bottom enclosure 127. The top and bottom enclosures 125 and 127 are snap fitted together. A sealant material such as bead of silicon may be applied to the lip of one or both of the top and bottom enclosures 125, 127 prior to joining them together so as to form a water-tight seal at the join between the top and bottom enclosure 127. This may beneficially protect against water ingress into the electronics module 100. The use of a two or more enclosures which are coupled together such as the top enclosure 125 and the bottom enclosure 127 is not required in all examples of the present disclosure. A single piece housing such as one which is overmoulded over the components of the module 100 is also within the scope of the present disclosure. Alternatively or additionally, the top enclosure 125 and the bottom enclosure 127 may be joined together by screws, sonic welding, glue or by any other means known to those skilled in the art.

The contact pads 103, 104 are formed of two separate pieces of conductive elastomeric material 103, 104 that form first and second flexible conductors 103, 104. The conductive elastomeric material used in this example is a conductive silicone rubber material, but other forms of conductive elastomeric material may be used. Beneficially, elastomeric material such as conductive silicone rubber can have an attractive visual appearance and may easily be moulded or extruded to have branded or other visual elements.

The elastomeric material is made conductive by distributing a conductive material into the elastomeric material. Conductive particles such as carbon black and silica are commonly used to form conductive elastomeric materials but the present disclosure is not limited to these examples. https://en.wikipedia.org/wiki/Conductive elastomer - cite note-4The contact pads 103, 104 may also comprise a 2D electrically conductive material such as graphene or a mixture or composite of an elastomeric material and a 2D electrically conductive material.

The contact pads 103, 104 define an external surface 155 that faces away from the bottom enclosure 127. The surface 155 is arranged to interface with an external component so as to couple signals between the external component and a controller of the wearable article. The external component may be a conductive region of the wearable article or a skin surface of the wearer amongst other examples. The surface 155 is textured to provide additional grip when positioned on the garment 200 or the skin surface. The texture may be, for example, a ribbed or knurled texture. The elastomeric material 103, 104 shown in the Figures has a ribbed texture. The contact pads 103, 104 may be flat and are not required to have a textured surface.

The electronics module 100 further comprises an interface 15 for coupling the electronics module 100 to a further device so as to charge a battery of the electronics module 100 and/or transfer data between the electronics module 100 and the further device. The interface 15 is a USB-C interface.

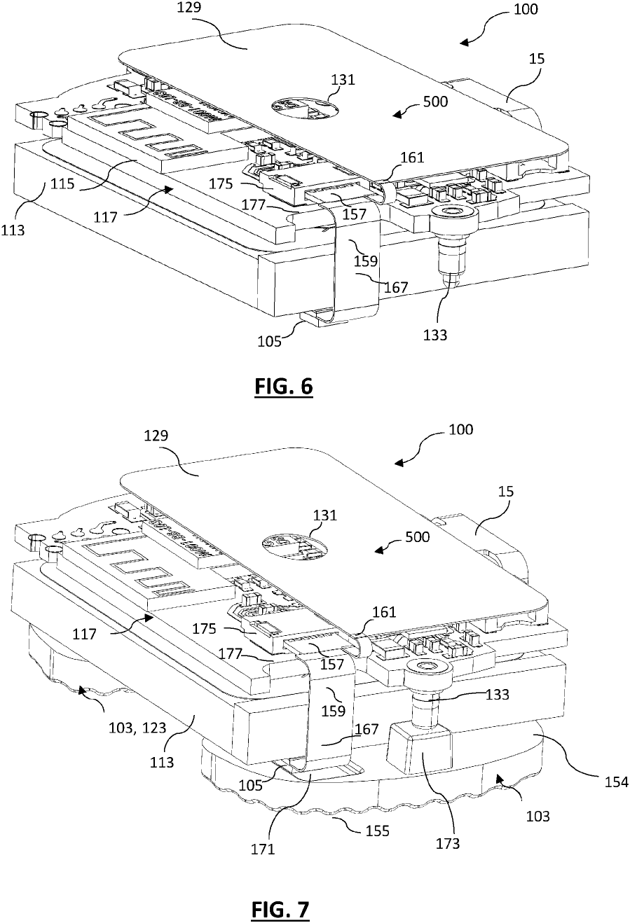

FIG. 6 shows the electronics module 100 with the housing 101 and the contact pads 103, 104 removed.

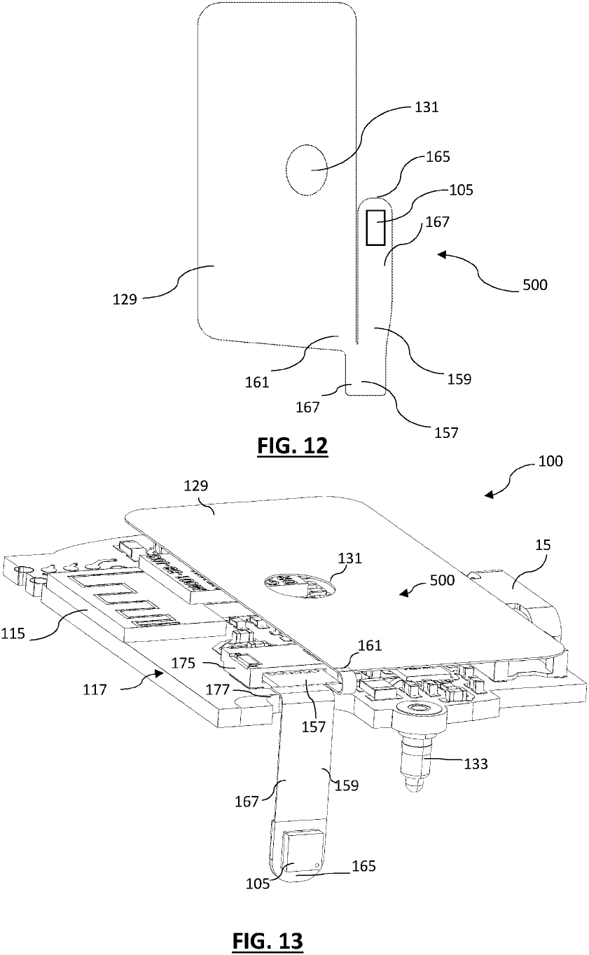

The electronics module 100 comprises a printed circuit board 117, a power source 113 in the form of a rechargeable battery 113 and a flexible electronics structure 500. The printed circuit board 117 is shown in isolation in FIG. 11. The flexible electronics structure 500 is shown in isolation in FIG. 12.

The processor 109 (FIG. 11), communicator 115 (FIGS. 6 and 11) and optional other electronics components such as light sources and sensors such as motion sensors are provided on the printed circuit board 117. The power source 113 is provided separately and below the printed circuit board 117.

FIGS. 6 and 12 show that the flexible electronics structure 500 comprises a flexible substrate material and electronics components 105, 129 incorporated into or otherwise mounted on the flexible substate material.

The flexible electronics structure 500 comprises a first electronics component 105 and a second electronics component 129. The first electronics component 105 in this example is a temperature sensor 105. The second electronics component 129 is a radio-frequency antenna 129 that functions as a communicator 129 forthe electronics module 100 (in addition to the communicator 115 in this example). The radio-frequency antenna 129 is a near-field communication (NFC) antenna 129. The present disclosure is not limited to these particular examples of electronics components 105, 129.

The radio-frequency antenna 129 may, for example, be any form of communication antenna. The antenna 129 may be a short-range communication antenna 129 arranged to transmit and/or receive data over a communication range of up to 50 metres, optionally up to 30 metres, optionally up to 10 metres, and optionally up to 1 metre. The short-range communication antenna may comprises one or more of a near field communication, NFC, wireless body area network, BAN, and a wireless personal area network, PAN, communication antenna. The short-range communication antenna may comprise one or more of a NFC, Bluetooth®, Bluetooth® Low Energy, Bluetooth® Mesh, Bluetooth® 5, Thread, Zigbee, IEEE 802.15.4, and Ant communication antenna.

The antenna 129 may be a medium-range communication antenna. The medium-range communication antenna may be arranged to transmit and/or receive data over a communication range of up to 200 metres, optionally up to 100 metres, optionally up to 50 metres, optionally up to 30 metres. The medium-range communication antenna may comprise one or more of a wireless near-me area network, NAN, a wireless local area network, WLAN, and a Wi-Fi communication antenna.

The antenna 129 may be a long-range communication antenna. The long-range communication antenna may be arranged to transmit and/or receive data over a communication range of over 200 metres, optionally over 100 metres, optionally over 50 metres. The long-range communication antenna may comprise one or more of a wireless metro-area network, WMAN, a wireless wide area network, WAN, a low power wide area network, LWAN, and a cellular antenna. The cellular antenna may be configured to transmit or receive data over one or more of a fourth generation (4G) LTE, LTE Advanced (LTE-A), LTE Cat-M1, LTE Cat-M2, NB-IoT, fifth generation (5G), sixth generation (6G), and/or any other present or future developed cellular wireless network. The antenna 129 may be a Global Navigation Satellite System, GNSS, receiver.

The antenna 129 is not required to be a communication antenna and may be a power receiving antenna for example.

The temperature sensor 105 is a discrete component that is mounted on the flexible substrate material. The antenna 129 is formed of traces of conductive material such as copper on the flexible substrate material.

The sensor and antenna 105, 129 are electrically connected to a shared interface region 157 by traces of conductive material such as copper on the flexible substrate material. In the shared interface region 157, an interface for the sensor 105 is provided proximate to the interface for the antenna 129.

The interface region 157 forms a single connector interface point for the flexible electronic structure 500 for connecting with the printed circuit board 117. The printed circuit board 117 comprises a connector interface 175 for connecting with the interface region 157.

Providing a shared interface region 157 reduces the number of connector interfaces 175 that are required on the printed circuit board 117. Ratherthan requiring separate connector interfaces 175 for the temperature sensor 105 and the antenna 129, a single connector interface 175 for both the temperature sensor 105 and the antenna 129 is provided. Reducing the number of connector interfaces 175 on the printed circuit board 117 is beneficial in reducing the overall size of the printed circuit board 117 and/or means that more electronics components can be provided on the printed circuit board 117 as less space is taken up by connector interfaces 175. Reducing the size of the printed circuit board 117 is generally beneficial for electronics modules 100 for wearable articles as it means that the electronics module 100 can be smaller and thus more discretely integrated into the wearable article.

The flexible substrate of the flexible electronics structure 500 comprises a first arm 159 and a second arm 161. The first arm 159 and second arm 161 are able to move relative to one another so as to be located at different positions in the electronics module 100.

The first arm 159 is an elongate strip of flexible substrate that terminates in an end region 165 in which the temperature sensor 105 is provided. The end region 165 is shaped to facilitate insertion of the first arm 161 through a further component such as a recess in the housing 101 or contact pad 103, 104 as explained in further detail below.

FIG. 6 shows the first arm 159 bent downwards and away from the interface region 157 to provide the temperature sensor 105 below the printed circuit board 117 and proximate to the bottom enclosure 127 (FIG. 5) of the housing 101. This arrangement means that the temperature sensor 105 is spaced apart from the printed circuit board 117 and positioned closer to a skin surface of the wearer when worn. Since the temperature sensor 105 is spaced apart from the printed circuit board 117 it is less affected by heat generated by the printed circuit board 117. Since the temperature sensor 105 is provided proximate to the bottom enclosure 127, it is provided closer to the skin surface when worn and can thus obtain a temperature reading which is a better reflection of the skin surface temperature.

The second arm 161 is bent upwards and away from the interface region 157 to provide the antenna 129 above the printed circuit board 117 and proximate to the top enclosure 125 (FIG. 4) of the housing 101. The NFC antenna 129 is positioned above the printed circuit board 117. The NFC antenna 129 is provided proximate to the top enclosure 125. The bottom enclosure 127 is closest to the body of the wearer in use and the top enclosure 125 is furthest away from the body of the wearer in use. Beneficially, providing the NFC antenna 129 proximate to the top enclosure 125 minimises the communication distance between the NFC antenna 129 and the mobile device 300.

The second arm 161 comprises an aperture 131. The conductive traces forming the radio-frequency antenna 129 are provided around the aperture 131. The radio-frequency antenna 129 may comprise a substantially spiral antenna coil. The NFC traces thus lie around an aperture 131 in the substrate to allow other devices or assemblies such as those located on the printed circuit board 117 to protrude through to the aperture 131, and the unobstructed transmission of light from a light source provided on the printed circuit board 117.

The first arm 159 comprises a first bend in the vicinity of the interface region 157 and a second bend in the vicinity of the temperature sensor 105. This arrangement means that the antenna 129 and temperature sensor 105 are parallel to one another and spaced apart from one another. The antenna 129 and temperature sensor 105 are separated by the battery 113 and printed circuit board 117.

FIGS. 7 to 9 show the electronics module 100 with the housing 101 removed. Conductors 133, 135 extend from the printed circuit board 117 so as to electrically connect the printed circuit board 117 to the contact pads 103, 104. The conductors 133, 135 in this example are springloaded pins 133, 135 which are also known as pogo-pins 133, 135.

In a preferred example, the pogo pins 133, 135 are suitable to be applied using a surface mount technology which lowers manufacturing costs. An example of such as pogo pin is the P70-2000045R pogo pin from Harwin PLC. Such surface mount suitable pogo pins may include additional locating pins for use in the surface mount process. These locating pins may, beneficially, provide additional structural support and reduce translational movement of the pogo pins relative to the printed circuit board 117.

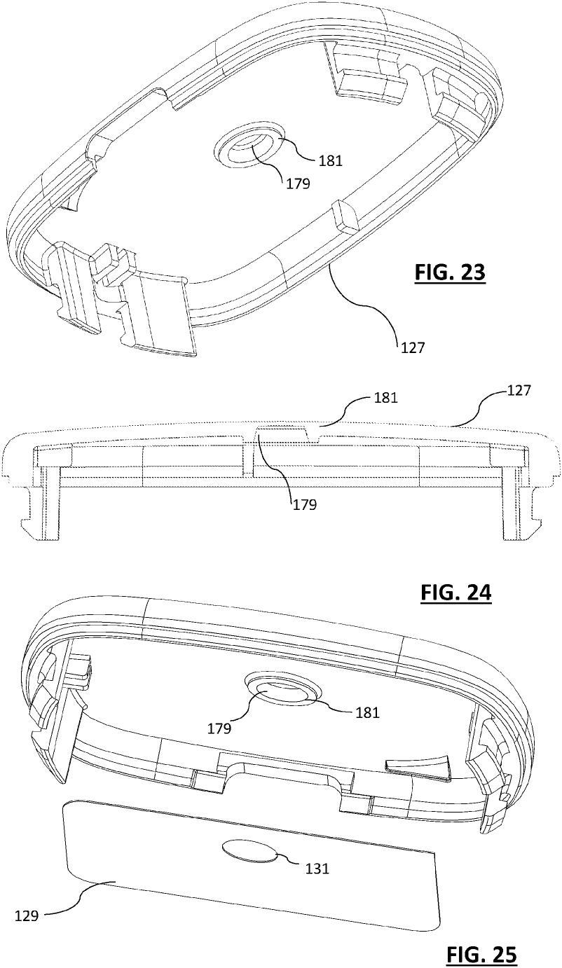

The contact pads 103, 104 comprises projections 173 extending from surface 154 and arranged to interface with pogo-pins 133, 135 so as to electrically connect the contact pads 103, 104 to the printed circuit board 117. The projections 173 may extend at least partially into the bottom enclosure 127 of the housing 101 such that the electrical contact between the contact pads 103, 104 is formed at least partially within the housing 101. FIG. 24 shows a close-up view of the inner side of the bottom enclosure 127. The projection 173 of the contact pad 103 extends through recess 19 in the bottom enclosure 127 so as to enter the internal side of the bottom enclosure 127 and contact the pogo-pin 133.

The contact pad 103 is shaped to accommodate the temperature sensor 105. In particular, the contact pad 103 comprises a recess 171 (FIGS. 7, 18 and 19) formed in the upper surface 154 of the contact pad 103. The upper surface 154 faces towards the bottom enclosure 127 in use. The recess 171 is sized to accommodate, at least partially, the temperature sensor 105. This arrangement enables the temperature sensor 105 to be protected by the contact pad 103. The temperature sensor 105 is placed in thermal contact with the contact pad 103 such that there is no air gap between the temperature sensor 105 and the contact pad 103. This helps ensure that a high quality signal is recorded by the temperature sensor 105. It will be appreciated that when the electronics component 105 is not a contact temperature sensor, thermal contact between the electronics component 105 and contact pad 103 is not necessarily required.

FIG. 9 shows that the contact pad 103 covers the temperature sensor 105 and pogo-pin 133 and protects these components against damage. The contact pad 104 covers the pogo-pin 134. The contact pads 103, 104 seal off the housing 101 and protect against water ingress into the housing 101.

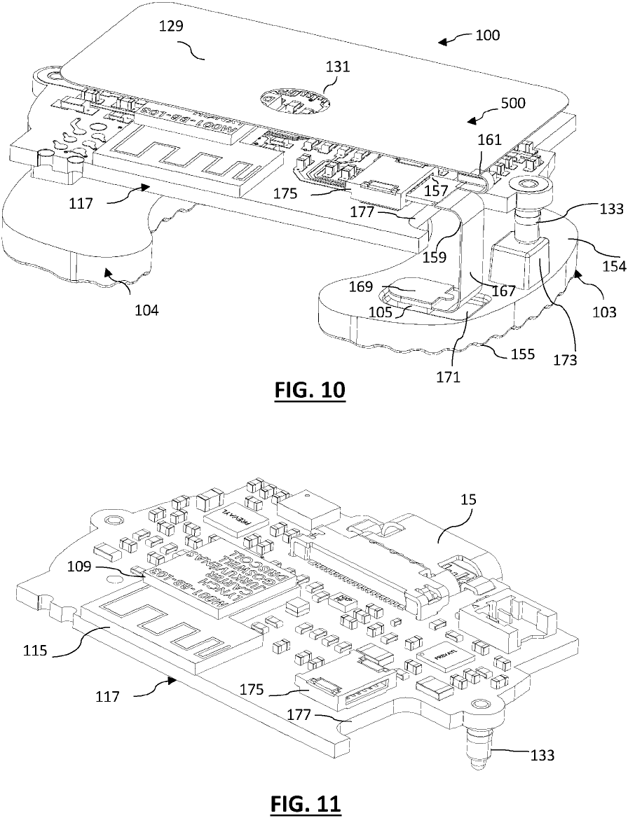

FIG. 10 shows the electronics module 100 with the housing 101 and power source 113 removed. FIG. 10 shows that an adhesive layer 169 applied to surface of flexible substrate in vicinity of temperature sensor 105 to enable temperature sensor 105 to be attached to the external surface of the bottom enclosure 127.

FIGS. 11 to 17 show a sequence of steps by which the electronics module 100 may be assembled according to aspects of the present disclosure.

The printed circuit board 117 (FIG. 11) and flexible electronics structure 500 (FIG. 11) are manufactured separately in this example, but in some examples the printed circuit board 117 and flexible electronics structure 500 may form a unitary printed circuit board structure such as a flexible circuit board structure 117, 500 or a rigid-flexible circuit board structure 117, 500.

The printed circuit board 117 is assembled using surface mount techniques and goes through a test stage. During the test stage, the printed circuit board 117 enters a jig where the flexible electronic structure 500 is attached to the printed circuit board 117.

This attachment is formed by connecting the interface region 157 (FIGS. 12 and 13) of the flexible electronic structure 500 to the connector interface 175 (FIGS. 11 and 13) of the printed circuit board 117. As the electronic components 105, 129 of the flexible electronic structure 500 share a common interface region 157, only a single electrical connection needs to be formed between the flexible electronic structure 500 and the printed circuit board 117. In other examples, a more permanent attachment between the printed circuit board 117 and the flexible electronics structure 500 may be formed. This may be achieved using hot bar soldering, for example. The connector interface 175 may be constructed differently when hot bar soldering is used.

A further mechanical attachment is achieved by an adhesive backing applied to the flexible substrate of the electronic structure 500. The adhesive backing is applied to the underside surface of the antenna 129 and enables the antenna 129 to be adhered to one or more components of the printed circuit board 117 (FIG. 13). The adhesive can help at least temporarily secure the antenna 129 to the printed circuit board 117 during assembly/test stages. The adhesive is not required in all examples of the present disclosure.

As shown in FIG. 13, the weight of the electronics component 105 causes the first arm 159 of the flexible electronic structure 500 to hang downwards. A stiffener material 167 applied to the first arm 159 can further help the first arm 159 hand downwards and restrict flapping or streaming movement of the first arm 159. This facilitates the assembly process as explained in further detail below. Moreover, the printed circuit board 117 has a cut-out region 177 in the vicinity of the connector interface 175. The cut-out region 177 helps the first arm 159 to hang downwards.

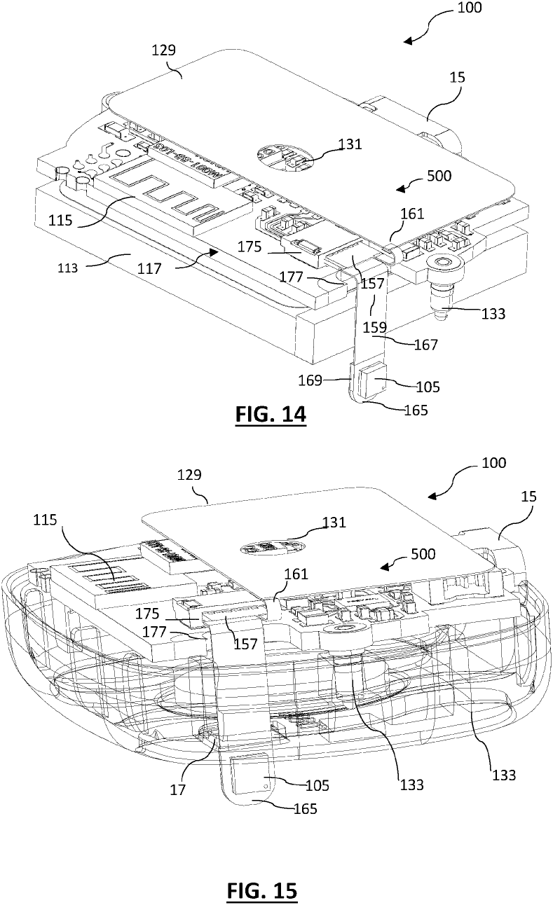

The printed circuit board/flexible electronic structure assembly 117, 500 then enters another jig where the power source 113 is electrically and mechanically coupled to the printed circuit board 117 (FIG. 14).Thejig helps ensure accurate placement of the battery 113 within the enclosure. A double-sided foam adhesive is used to secure the battery 113 to the printed circuit board 117 so as to counter effects such as expansion of the battery 113 over time.

The printed circuit board 117, flexible electronic structure 500, and battery 113 are initially connected together to form an assembly before being disposed within the housing 101. This enables the components to be positioned within the housing 101 using a simple procedure with a limited number of steps.

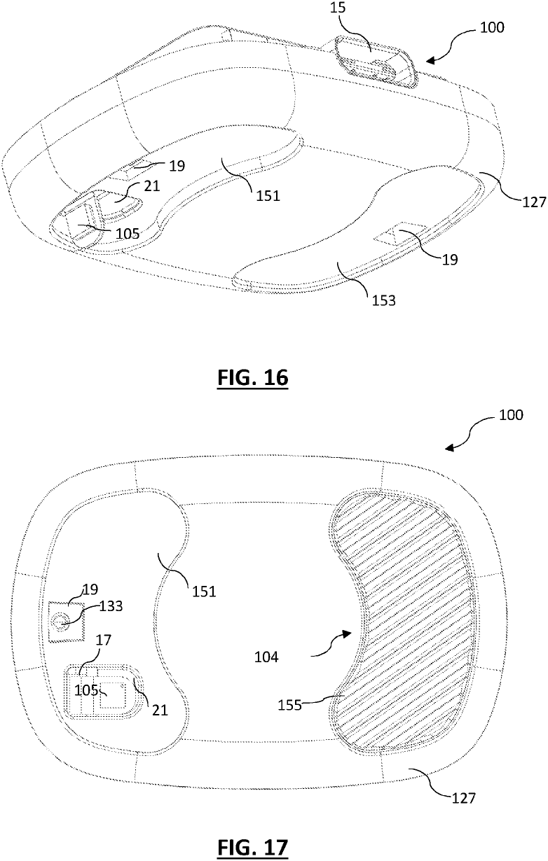

The assembly 117, 500, 113 is then lowered into the bottom enclosure 127 of the houisng 101 from above (FIGS. 15 and 16). The arm 159 of the flexible electronic structurer 500 hangs downwards due to the weight of the temperature sensor 105 and the stiffener material 167. The stiffener material 167 may be provided in the form of a stiffener layer 167. The stiffener material 167 helps the first arm 159 straight during insertion into the bottom enclosure 127.

Stiffener layers may also be provided in the area under the temperature sensor 105 and the connector interface 157 to strengthen these areas and protect them from damage during insertion. The stiffener layers 167 may be formed from polyimide material and typically have a thickness of between 0.1 mm and 0.3 mm, preferably 0.2 mm.

As the assembly 117, 500, 113 is lowered into the bottom enclosure 127, the temperature sensor 105 of the flexible electronic structure 500 passes through the opening 17 in the bottom enclosure 127 such that the temperature sensor 105 passes through the bottom enclosure 127 from inside the bottom enclosure 127 to outside the bottom enclosure 127. The end region 165 of the arm 159 is rounded to help facilitate the passing of the arm 159 through the opening 17.

Once the assembly 117, 500, 113 is firmly inserted into the bottom enclosure 127, an adhesive tape carrier film covering an adhesive layer 169 (FIG. 10) on the underside of the temperature sensor 105 is removed and the first arm 159 is bent by approximately 90 degrees to adhere the flexible substrate to the underside of the bottom enclosure 127 (FIG. 17). The bottom enclosure 127 has a recess 21 which accommodates the flexible substrate. The temperature sensor 105 to the external surface of the bottom enclosure 127 so that the temperature sensor 105 faces away from the bottom enclosure 127. The recess 21 on the underside of the bottom enclosure 127 is slightly larger than needed, this is to account for any human error in terms of placement and potential tolerance issues with the flexible substrate.

Once the temperature sensor 105 is in place, the contact pad 103 is then pushed into its corresponding recess 151 in the bottom enclosure 127 (FIG. 17). The projection 173 of the contact pad 103 extends into the opening 19 so as to make electrical contact with the pogo-pin 133. This enables the contact pad 103 to make electrical contact with the pogo pin 133 in addition to thermal contact with the temperature sensor 105. The contact pad 104 is also pushed into its corresponding recess 153 such that its projection extends through opening 19 so as to make contact with the pogo-pin 135. FIG. 17 shows the contact pad 104 already attached to the bottom enclosure 127.

Double sided-adhesive layers are used to adhere the contact pads 103, 104 to the outer surface of the bottom enclosure 127. The adhesive layers may be adhesive transfer tape such as adhesive transfer tape 467 and adhesive transfer tape 468 provided by 3 M. The contact pads 103, 104 have a push tight in the recesses 151, 153 to help ensure that no dust or debris is able to enter the electronics module 100. Beneficially, in this arrangement, the contact pads 103, 104 seal the openings 17, 19 in the bottom enclosure 127 and thus prevent water ingress into the electronics module 100. Therefore, the electronics module 100 is waterproof while still enabling electrical connection between internal components of the electronics module and external components.

The top enclosure 125 is attached to the bottom enclosure 127 such as by using a snap-fit mechanism. The snap-fit between the top and bottom enclosures 125, 127 helps ensure that the pogo pins 133, 135 are under and constant and even pressure, and is thus in constant contact with the contact pads 103, 104. The top enclosure 125 may comprise mounting pins to help apply pressure to the printed circuit board 117 and thus to the pogo pins 133,135.

The present disclosure is not limited to pogo pins. Other forms of conductor and particularly force-biased conductors may be used to connect the printed circuit board 117 to the contact pads 103, 104. For example, conductive leaf springs may be used.

Moreover, other processes could be used to connect the printed circuit board 117 to the contact pad 103, 104 such as by soldering the connections, terminating the printed circuit board 117 by means of a fixing such as a screw or bolt, or by crimping the contact pads 103, 104 to the printed circuit board 117, These approaches are generally less preferred as they are more costly and labour intensive than the implementations described above, but are still within the scope of the present disclosure.

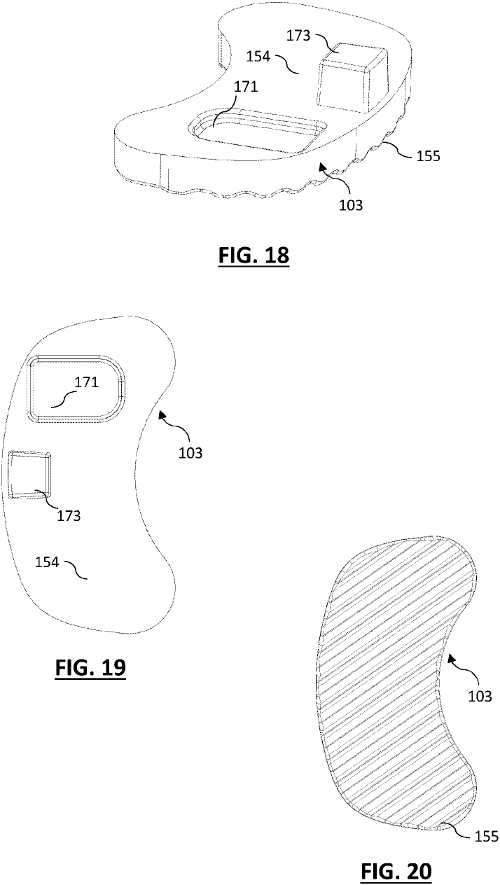

FIGS. 18 to 20 show an example contact pad 103 in isolation. The contact pad 103 is useable with the electronics module 100 of FIGS. 4 to 17 but is not limited for use with such electronics modules 100. For example, the contact pad 103 may not be attached to a housing 101. The contact pad 103 may be provided as a stand-alone component or may be incorporated directly into a wearable article such as a wristband, chestband, or arm band of other form of garment.

FIG. 12 shows the flexible electronic structure 500 in isolation. The flexible electronic structure 500 is useable with the electronics module 100 of FIGS. 4 to 17 but is not limited for use with such electronics modules 100. The flexible electronic structure 500 may be a stand-alone structure or may be incorporated in other forms of electronic devices such as other forms of wearable devices or non-wearable devices.

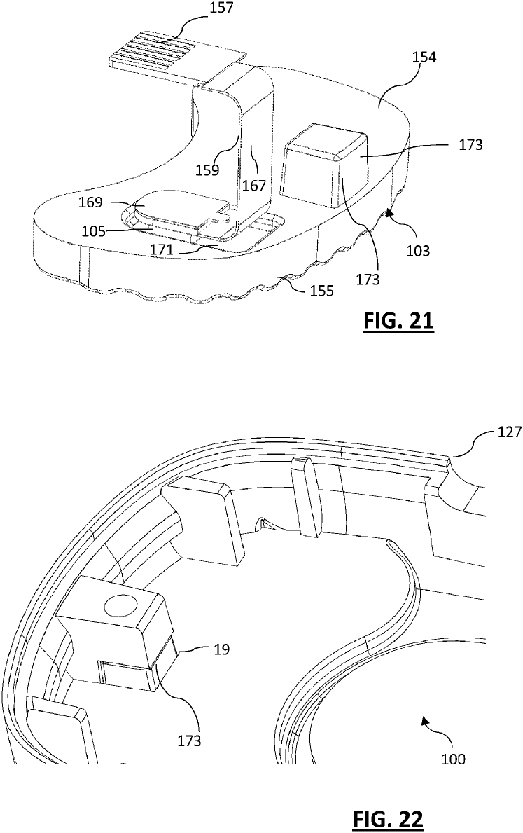

FIG. 21 shows a contact pad assembly in isolation. The contact pad assembly is useable with the electronics module 100 of FIGS. 4 to 17 but is not limited to use with such electronics modules 100. The contact pad assembly comprises a contact pad 103 and an electronics component 105 located with the contact pad 103. The contact pad 103 may be the contact pad of FIGS. 18 to 20.

The electronics component 105 is in contact with the contact pad 103. The contact is a thermal contact in this example, but the sensor 105 may also be in electrical contact with the contact pad 103 in certain applications.

The contact pad 103 is shaped to accommodate the electronics component 105. In particular, the contact pad 103 comprises recess 171 which is sized to receive at least part of the electronics component 105. The electronics component 105 is therefore partially located within the recess 171. In other examples, the electronics component 105 may be attached to the contact pad 103 such as by being overmoulded with the contact pad 103.

The contact pad assembly further comprises a connector 159 arranged to connect the electronics component 105 with a further electronics component of the wearable article. The connector 159 is a flexible substrate on which the electronics component 105 is deposited. The connector 159 has an interface region 157 for connecting with the further electronics component of the wearable article. The connector 159 corresponds to the first arm 159 of the flexible electronics assembly 500 of FIG. 12. In this example, the second arm 161 is not required. That is, the contact pad assembly is not required to include the second arm 161 of the flexible electronics assembly on which the second electronics component 129 (e.g. NFC antenna) is provided.

The flexible substrate comprises a stiffener material 167 in the vicinity of any or all of the sensor 105, the interface region 157 and the length of flexible substrate between the electronics component 105 and the interface region.

The electronics component 105 in this example comprises a sensor and, in particular, comprises a temperature sensor. In particular a contact temperature sensor 105 such as a thermistor or thermocouple. Other examples of sensor 105 include pressure sensors, humidity sensors, PPG sensors, magnetometers, infrared temperature sensors, capacitive sensors, vibration sensors, gas sensors, infrared sensors for the transmission and/or receiving data, force sensitive resistors, radar and lidar. The present construction is beneficial for magnetometers as the construction enables the magnetometers to be spaced as far away from electronics within the module as possible. The electronics component 105 is not limited to sensors and includes other forms of electronics components 105 such as haptic feedback units.

FIGS. 23 to 25 show the inner side of the top enclosure 125. The inner side of the top enclosure 125 comprises a region of localised thinning 179. The region of localised thinning 179 is an area of the top enclosure 125 with a thinner wall than other parts of the top enclosure 125. The region of localised thinning 179 is substantially transparent or translucent such that a light source positioned in the housing 101 may emit light therethrough that is visible outside of the electronics module 100.

The region of localised thinning 179 is bounded by a region of increased wall thickness 181. The region of increased wall thickness 181 is an area of the top enclosure 125 with a thicker wall than other parts of the top enclosure 125. The region of increased wall thickness 181 supports the region of localised thinning 179 and also acts to stop light from bleeding out of the region of localised thinning 179. This means that a point of light is seen outside of the electronics module 100 rather than diffuse light.

The region of localised thinning 179 and the region of increased wall thickness 181 are made from the same material as the rest of the top enclosure 125. That is, the top enclosure 125 can be constructed as one piece using an injection moulding process or similar. A separate light pipe or a separate material region in the top enclosure 125 is not required. This simplifies the construction of the top enclosure 125.

The region of localised thinning 179 in this example is in the form of an ellipse. The region of localised thinning 179 is located in a central region of the top enclosure 125. The ellipse in this example has a major axis of 4 mm and a minor axis of 3 mm. The present disclosure is not limited to this example, and any dimension of ellipse and other shapes beyond ellipses are within the scope of the present disclosure.

The region of localised thinning 179 may have an area of at least 0.3 mm², at least 10 mm², at least 20 mm², at least 30 mm², at least 40 mm², at least 50 mm², at least 100 mm², at least 150 mm², at least 250 mm², at least 500 mm², or at least 1000 mm². The region of localised thinning 179 may have an area of less than 1500 mm², less than 1000 mm², less than 500 mm², less than 250 mm², less than 150 mm², less than 100 mm², less than 50 mm², less than 40 mm², less than 30 mm², less than 20 mm², or less than 10 mm².

The wall thickness in the region of localised thinning 179 is generally between 90% and 10% of the wall thickness of the remainder of the top enclosure 125. The wall thickness in the region of localised thinning 179 may be less than 90%, less than 80%, less than 70%, less than 60%, less than 50%, less than 40%, less than 30%, less than 20% or less than 10% of the wall thickness of the remainder of the top enclosure 125. The wall thickness in the region of localised thinning 179 may be more than 10%, more than 20%, more than 30%, more than 40%, more than 50%, more than 60%, more than 70%, or more than 80% of the wall thickness of the remainder of the top enclosure.

The wall thickness in the region of localised thinning 179 may be between 0.05 mm and 0.5 mm, between 0.05 mm and 0.4 mm, between 0.05 mm and 0.3 mm, between 0.05 mm and 0.2 mm, or between 0.05 mm and 0.1 mm. The wall thickness in the region of localised thinning 179 may be between 0.1 mm and 0.5 mm, between 0.2 mm and 0.5 mm, between 0.3 mm and 0.5 mm, between 0.4 mm and 0.5 mm.

The present disclosure is not limited to any particular wall thickness. Generally, the wall thickness is dependent on the size of the region of localised thinning 179. A larger region of localised thinning 179 will generally require a thicker wall thickness.

The wall thickness in the region of increased wall thickness 181 is between 0.1 mm and 1.5 mm. The wall thickness in the region of increased wall thickness 181 may be between 0.2 mm and 1.5 mm, between 0.3 mm and 1.5 mm, between 0.4 mm and 1.5 mm, between 0.5 mm and 1.5 mm, between 0.6 mm and 1.5 mm, between 0.7 mm and 1.5 mm, between 0.8 mm and 1.5 mm, between 0.9 mm and 1.5 mm, between 1.0 mm and 1.5 mm, between 1.1 mm and 1.5 mm, between 1.2 mm and 1.5 mm, between 1.3 mm and 1.5 mm, or between 1.4 mm and 1.5 mm. The wall thickness in the region of increased wall thickness 181 may be between 0.1 mm and 1.4 mm, between 0.1 mm and 1.3 mm, between 0.1 mm and 1.2 mm, between 0.1 mm and 1.1 mm, between 0.1 mm and 1.0 mm, between 0.1 mm and 0.9 mm, between 0.1 mm and 0.8 mm, between 0.1 mm and 0.7 mm, between 0.1 mm and 0.6 mm, between 0.1 mm and 0.5 mm, between 0.1 mm and 0.4 mm, between 0.1 mm and 0.3 mm, or between 0.1 mm and 0.2 mm.

The region of increased wall thickness 181 is not required in all examples. But can be beneficial in providing additional framing and support for the region of localised thinning 179 and to stop or reduce the diffusion of light.

FIG. 25 shows that the region of localised thinning 179 is aligned with the aperture 131 of the NFC coil 129. The aperture 131 has a similar shape and is just larger than the region of increased wall thickness 181 such that when assembled, the region of increased wall thickness 181 extends partly through the aperture 131 to help locate the NFC coil 129 and hold the NFC coil 129 in fixed position in relation to the top enclosure 125.

Since the aperture 131 is aligned with the region of localised thinning 179, light emitted by a light source of the printed circuit board 117 is able to pass through the aperture 131 and the region of localised thinning 179. The light is able to indicate, for example, the location of the antenna 129 in the electronics module 200 so as to indicate to a user where to tap their mobile device 300 for Bluetooth pairing for example.

The region of increased wall thickness 181 may be sized lock into aperture 131 to hold the NFC coil 129 in place. This may help facilitate the assembly of the electronics module 100.

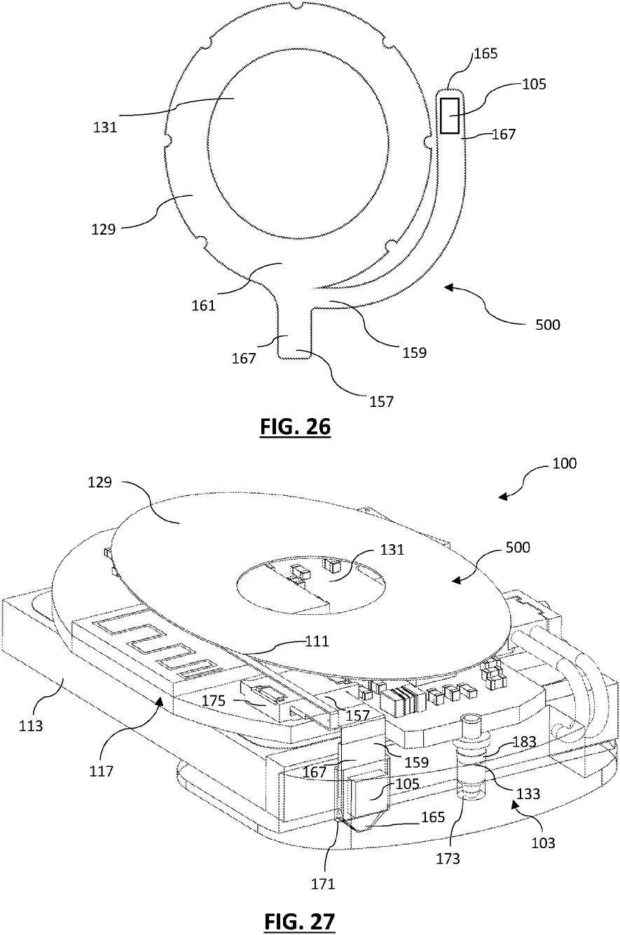

Referring to FIG. 26, there is shown another example of flexible electronic structure 500 according to aspects of the present disclosure.

Referring to FIG. 27, there is shown another example electronics module 100 according to aspects of the present disclosure. The housing 101 is not visible in this example.

In this example, the electronics component 105 is disposed within a recess 171 of the contact pad 103. The electronics component 105 extends downwards into the recess 171 and is not attached to the underside surface of the bottom enclosure 127 of the housing 101. Instead, the electronics component 105 is held in place by the recess 171. During assembly, the electronics component 105 is pushed into the recess 171.

The area around the electronics component 105 has a stiffener material 167 to protect the electronics component 105 during insertion into the recess 171. The stiffener material 167 helps keep the first arm 159 straight during the insertion into the recess 171.

The end region 165 of the first arm 159 is shaped like an arrow to help guide the electronics component 105 into the recess 171. The recess 171 in the contact pad 103 has a similar profile to the first arm 159. This helps protect the electronics component 105 if it is pushed too far into the recess 171 as the arrow shape will take the brunt of the impact/damage.

The recess 171 in the contact pad will also be slightly undersized to ensure a push tight fit. This will also help with maximising contact surface area between the contact pad 103 and the electronics component 105.

The contact pad 103 is also shaped to accommodate the pogo-pin 133 by having a recess 173 for receiving the pogo-pin 133. The pogo-pin 133 has a cylindrical aperture allowing for it to be partially recessed into the contact pad 103. The pogo-pin 133 further has a barbed region 183 to help mechanically and electrically secure the pogo-pin 133 in the recess 173.

The flexible electronics structure 500 may have a similar barbed region to facilitate the retention of the electronics component 105 in the recess 171. This is particularly beneficial when a housing is not provided for the electronics module 100 or where the conductive pad assembly comprising the conductive pad and the electronics component 105 are spaced apart from the housing 101 of the electronics module100.

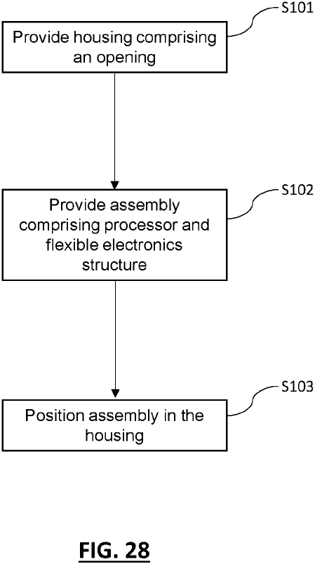

Referring to FIG. 28, there is shown a flow diagram for an example method of assembling an electronics module 100 according to aspects of the present disclosure. Step S101 of the method comprises providing a housing comprising an opening. Step S102 of the method comprises providing an assembly comprising a processor and a flexible electronics structure comprising a flexible substrate on which an electronics component is provided, wherein the electronics component is communicatively connected to the processor. Step S103 of the method comprises positioning the assembly in the housing such that the flexible substrate extends through the opening in the housing, the electronics component is located at least partially outside of the housing, and the processor is located within the housing.

The electronics modules 100 of the present disclosure are able to be manufactured in a simple and cost effective process. Generally, separate components of the electronics module 100 are able to be manufactured separately and then assembled together. For example, the printed circuit board 117 and pogo pins 113, 135 may be assembled together. Separately, the housing 125, 127 may be manufactured using techniques such as injection moulding. Separately still, the flexible electronic structure 500 may be manufactured. Separately still, the conductive material 121, 123 may be manufactured. These sub-assemblies may be manufactured at different specialised manufactures and subsequently assembled in a single location.

The electronics modules of the present disclosure 100 are not limited to one electronics component 105. For example, both the contact pad 103 and the contact pad 104 may accommodate an electronics component 105.

While the examples show electronics modules 100 with two contact pads 103, 104 it will be appreciated that the present disclosure is not limited to any particular number of contact pads 103, 104. One contact pad may be provided. Two or more contact pads may be provided. The number of contact pads will depend on the number of terminals in the garment to be connected to. For example, there may be 4, 6 or 10 contact pads. It will be appreciated that additional flexible conductors may be electrically connected to the printed circuit board through the use of additional pogo pins 133, 135 or other conductors.

In some examples, the wearable article may an in-ear headphone. The antenna coil 129 may be an NFC coil 129 used for Bluetooth® pairing of the headphone to a mobile device. The sensor 105 may be a temperature sensor 105 for performing in-ear temperature measurements. The contact pad may be the elastomeric covering of the in-ear headphone.

In the present disclosure, the electronics module may also be referred to as an electronics device or unit. These terms may be used interchangeably.

At least some of the example embodiments described herein may be constructed, partially or wholly, using dedicated special-purpose hardware. Terms such as ‘component’, ‘module’ or ‘unit’ used herein may include, but are not limited to, a hardware device, such as circuitry in the form of discrete or integrated components, a Field Programmable Gate Array (FPGA), programmable System on Chip (pSoC), or Application Specific Integrated Circuit (ASIC), which performs certain tasks or provides the associated functionality. In some embodiments, the described elements may be configured to reside on a tangible, persistent, addressable storage medium and may be configured to execute on one or more processors. These functional elements may in some embodiments include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. Although the example embodiments have been described with reference to the components, modules and units discussed herein, such functional elements may be combined into fewer elements or separated into additional elements. Various combinations of optional features have been described herein, and it will be appreciated that described features may be combined in any suitable combination. In particular, the features of any one example embodiment may be combined with features of any other embodiment, as appropriate, except where such combinations are mutually exclusive. Throughout this specification, the term “comprising” or “comprises” means including the component(s) specified but not to the exclusion of the presence of others.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1-23. (canceled)

24. An electronics module for a wearable article comprising:
a housing comprising an opening;
a processor;
a sensor arranged to monitor a property of a wearer of the wearable article;
a flexible electronics structure comprising a flexible substrate on which the sensor is provided, wherein the sensor is communicatively connected to the processor,
a contact pad; and
a conductor that extends from the processor and contacts the contact pad so as to electrically connect the processor to the contact pad, wherein the flexible substrate extends through the opening in the housing such that the sensor is located at least partially outside of the housing, and the processor is located within the housing, and
wherein the sensor is sandwiched between the contact pad and the housing.

25. The electronics module according to claim 24, wherein the contact pad is shaped to accommodate at least part of the sensor.

26. The electronics module according to claim 25, wherein the contact pad comprises a recess sized to receive at least part of the sensor.

27. The electronics module according to claim 24, wherein the contact pad is in thermal contact with the sensor.

28. The electronics module according to claim 24, wherein the housing comprises an opening for receiving at least part of the contact pad.

29. The electronics module according to claim 24, wherein the housing comprises a first enclosure and a second enclosure which are connected to one another.

30. The electronics module according to claim 29, wherein the first enclosure and the second enclosure are connected to one another using a snap-fit mechanism.

31. The electronics module according to claim 24, wherein the sensor is attached to an external surface of the housing.

32. The electronics module according to claim 24, wherein the sensor is located in a recess provided in an external surface of the housing.

33. A method of assembling an electronics module for a wearable article, the method comprising:
providing a housing comprising an opening;
providing an assembly comprising a processor, a conductor, a sensor arranged to monitor a property of a wearer of the wearable article, and a flexible electronics structure comprising a flexible substrate on which the sensor is provided, wherein the sensor is communicatively connected to the processor;
positioning the assembly in the housing such that the flexible substrate extends through the opening in the housing, the sensor is located at least partially outside of the housing, and the processor is located within the housing; and
attaching a contact pad to an external surface of the housing such that the sensor is sandwiched between the contact pad and the housing and the contact pad contacts the conductor so that it is electrically connected to the processor.

34. The method according to claim 33, wherein the flexible electronics structure comprises a connector interface region which is communicatively connected to the processor and an end region on which the sensor is provided.

35. The method according to claim 34, wherein the end region is able to hang downwards due to gravity.

36. The method according to claim 34, wherein positioning the assembly in the housing comprises lowering the assembly into the housing such that the end region of the flexible electronics structure passes through the opening in the housing.

37. The method according to claim 33, wherein providing the housing comprises providing a first enclosure comprising the opening.

38. The method according to claim 37, further comprising attaching a second enclosure to the first enclosure to form an enclosed space in which the processer is located.

39. The method according to claim 33, further comprising providing a power source.

40. The method according to claim 39, further comprising attaching the power source to the processor.

41. The method according to claim 40, wherein the attaching comprises electrically and mechanically attaching the power source to the processor.

42. The method according to claim 40, wherein the power source is attached to the processor prior to positioning the assembly in the housing.

43. The method according to claim 33, further comprising locating the sensor in a recess of the contact pad sized to receive at least part of the sensor.

44. The method according to claim 33, further comprising attaching the sensor to an external surface of the housing.

45. The method according to claim 33, wherein the sensor is located in a recess provided in an external surface of the housing.
